(19) United States
(12) Patent Application Publication
(10) Pub. No.: US 2026/0090748 A1
(43) Pub. Date: Apr. 2, 2026

(54) LIFE ATTITUDE TEST METHOD AND SYSTEM

(71) Applicant: Song LEE, Jeju-si (KR)

(72) Inventor: Song LEE, Jeju-si (KR)

(21) Appl. No.: 18/952,234

(22) Filed: Nov. 19, 2024

(30) Foreign Application Priority Data
Sep. 30, 2024 (KR) 10-2024-0132492

(51) Int. Cl. A61B 5/16; G16H 10/20

(52) U.S. Cl. CPC A61B 5/165; G16H 10/20

(57) ABSTRACT

According to an aspect of the present invention, there is provided a life attitude test method, the life attitude test method being performed by a test server, the life attitude test method including: providing a plurality of questions for each of a plurality of evaluation factors, including a vertical life, a horizontal life, a gaslighting state, and an enlighting state, to a user terminal; receiving user-entered information for each of the plurality of questions for each of the evaluation factors from the user terminal; and calculating total scores for the respective evaluation factors based on the information received from the user terminal.

### CROSS-REFERENCE TO RELATED APPLICATION

This application claims the benefit of Korean Patent Application No. 10-2024-0132492 filed on Sep. 30, 2024, which is hereby incorporated by reference herein in its entirety.

### BACKGROUND

### 1. Technical Field

The present invention relates to a life attitude test method and system, and more specifically, to a method and system that enable the value of a user's life and a self-esteem state to be tested.

### 2. Description of the Related Art

In today's society, informatization and industrialization are rapidly being performed in various fields. People today are feeling serious confusion about values in which excessive competition and achievement are emphasized, and at the same time, are suffering from mental health problems attributable to anxiety and alienation in daily life due to a lack of morality and humanity.

Some children may have difficulty developing a healthy sense of self due to frequent absence of conversations caused by their parents' busy lives, the absence of parents at home, or bullying from neighbors or friends. Furthermore, some children or adults may not be friendly to society and may have aggressive attitudes toward society.

Various people with anxious or aggressive tendencies tend to have difficulty expressing their psychological states.

Conventionally, there has been used a method in which paper psychological tests are utilized and psychological states are analyzed or treated or academic achievement is improved based on the results of the psychological tests. However, this conventional method has problems in that users are not interested in expressing their psychological states because it requires passive responses to standardized questions on the paper psychological tests and in that appropriate psychological treatment cannot be performed because appropriate psychological understanding cannot be achieved.

Recently, there have been an increasing number of psychological treatment applications that provide counseling treatment services on mobile platforms. However, most of the current related app services lack psychiatric or clinical psychological expertise, or users find them uncomfortable. Accordingly, people are not interested in the app services themselves, so that they have a disadvantage in that the usage rate thereof is generally low.

In addition, as interest in the degree of satisfaction with life and the degree of happiness has increased and also interest in gaslighting has increased recently, there is a demand for a test related to these issues.

### SUMMARY

Objects of the present invention are intended to overcome the problems of the above-described conventional art.

An object of the present invention is to enable the value of a user's life and a self-esteem state to be measured systematically in an easy way.

An object of the present invention is to enable the evaluation of self-protection ability, level of satisfaction with life, etc.

Objects of the present invention are not limited to the objects mentioned above, and other objects not mentioned may be clearly understood from the following description.

According to an aspect of the present invention, there is provided a life attitude test method, the life attitude test method being performed by a test server, the life attitude test method including: providing a plurality of questions for each of a plurality of evaluation factors, including a vertical life, a horizontal life, a gaslighting state, and an enlighting state, to a user terminal; receiving user-entered information for each of the plurality of questions for each of the evaluation factors from the user terminal; and calculating total scores for the respective evaluation factors based on the information received from the user terminal.

Providing the plurality of questions for each of the evaluation factors to the user terminal may include providing the plurality of questions for each of the evaluation factors that are mixed in a random order.

The life attitude test method may further include providing the name of an evaluation factor having a larger one between the total score for the vertical life and the total score for the horizontal life and the name of an evaluation factor having a larger one between the total score for the gaslighting state and the total score for the enlighting state as information about a user's self structure to the user terminal.

According to one embodiment of the present invention, it may be possible to systematically measure the value of a user's life and a self-esteem state in an easy way.

According to one embodiment of the present invention, it may be possible to evaluate one's self-protection ability, the degree of satisfaction with life, etc.

### BRIEF DESCRIPTION OF THE DRAWINGS

The above and other objects, features, and advantages of the present invention will be more clearly understood from the following detailed description taken in conjunction with the accompanying drawings, in which:

FIG. 1 is a diagram schematically showing the configuration of a life attitude test system according to one embodiment of the present invention;

FIG. 2 is a block diagram illustrating the detailed operation and internal configuration of a test server according to one embodiment of the present invention;

FIG. 3 is a diagram showing an example of an analysis result graph provided to a user terminal according to one embodiment of the present invention; and

FIG. 4 shows the types of graph forms according to test results according to one embodiment of the present invention.

### DETAILED DESCRIPTION

The detailed description of the present invention to be set forth below refers to the accompanying drawings that illustrate specific embodiments in which the present invention may be practiced. These embodiments are described in sufficient detail to enable a person skilled in the art to practice the present invention. It should be understood that the various embodiments of the present invention are different from each other but are not necessarily mutually exclusive. For example, specific shapes, structures and characteristics described herein are related to exemplary embodiments and may be implemented as other embodiments without departing from the spirit and scope of the present invention. Furthermore, it should be understood that the locations or arrangements of individual components within each disclosed embodiment may be changed without departing from the spirit and scope of the present invention. Accordingly, the detailed description to be set forth below is not taken to be limiting, and the scope of the present invention is defined only by the appended claims, along with the overall range equivalent to what the attached claims claim, when appropriately stated. In the drawings, like reference numerals refer to the same or like functions in various aspects.

In order to enable a person having ordinary knowledge in the art to which the present invention pertains to easily implement the present invention, preferred embodiments of the present invention will be described in detail with reference to the accompanying drawings below.

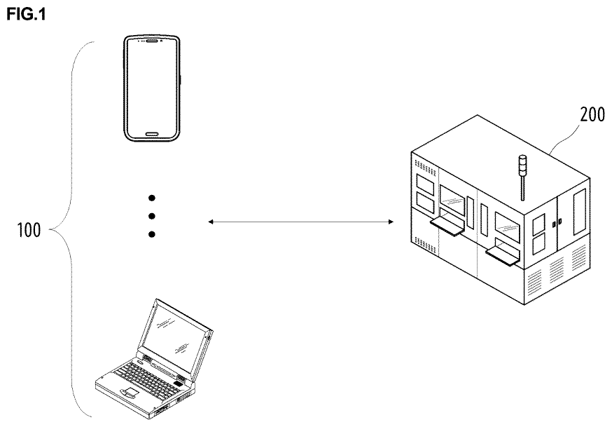

FIG. 1 is a diagram schematically showing the configuration of a life attitude test system according to one embodiment of the present invention.

Referring to FIG. 1, a life attitude test system according to one embodiment may include user terminals 100 and a test server 200.

The user terminals 100 and the test server 200 may communicate with each other over a communication network, e.g., a mobile communication network, a local area network (LAN), a metropolitan area network (MAN), a wide area network (WAN), a world wide web (WWW), and/or a wireless communication network (Wi-Fi).

Each of the user terminals 100 has a computation function, and may be implemented in any form as long as it is a device that can communicate with the outside. For example, the user terminal 100 may be implemented as a smartphone, a tablet personal computer (PC), a desktop, or a laptop notebook, but is not limited thereto.

The user terminal 100 performs the function of receiving test-related questions from the test server 200 and transmitting responses, input by a user, to the test server 200. Furthermore, the user terminal 100 receives a test result from the test server 200 and displays it in a form that the user can view.

The test server 200 transmits questions related to attitude toward life to the user terminal 100, and derives a test result based on responses transmitted from the user terminal 100.

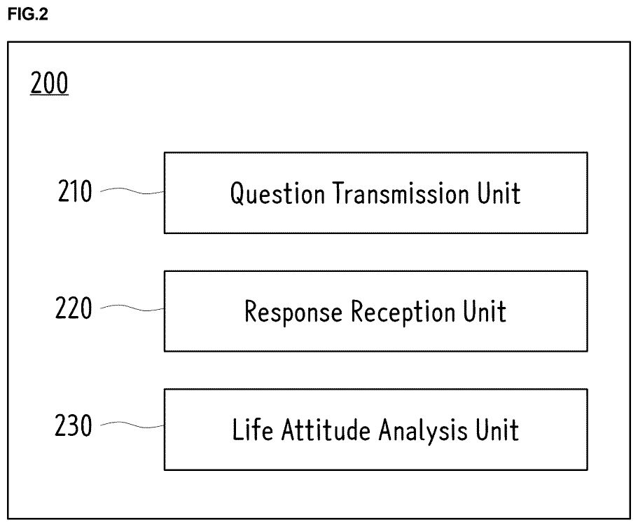

FIG. 2 is a block diagram illustrating the detailed operation and internal configuration of a test server according to one embodiment of the present invention.

Referring to FIG. 2, the test server 200 according to one embodiment may include a question transmission unit 210, a response reception unit 220, and a life attitude analysis unit 230.

The question transmission unit 210, the response reception unit 220, and the life attitude analysis unit 230 may be program modules or hardware that can communicate with an external device. These program modules or hardware may be included in the test server 200 or another device capable of communicating therewith in the form of an operating system, an application program module, or another program module, and may be physically stored on various known memory devices. Meanwhile, these program modules or hardware include, but are not limited to, routines, subroutines, programs, objects, components, data structures, etc. that perform specific tasks to be described below or execute specific abstract data types according to the present invention.

The question transmission unit 210 transmits questions for a life attitude test to the user terminal 100.

According to one embodiment, the question transmission unit 210 provides the user terminal 100 with 40 items listed in the following order.
1 I set a goal and try to achieve it.
2 I know what I like.
3 I have ambition for success.
4 I try to eat food that is good for my body.
5 I work excessively hard, and people around me ask why I do so hard.
6 I can clearly and politely defend my rights when I am treated unfairly.
7 I feel trust in my relationships with people (a lover, family, friends, etc.) I love.
8 The most precious moment for me is the present moment.
9 The future regarding how I will live is important to me.
10 I often hear people around me say, “You are being exploited; be careful” (because of concerns about a relationship with people close to me).
11 I sometimes feel anxious that I always have to do something.
12 I love myself even when I do not do well at something.
13 I care about others excessively much more than I do about myself.
14 I deal well with stress.
15 I love myself the way I am.
16 Sometimes, I think people treat me rudely.
17 I try to get along well with people.
18 People treat me with respect.
19 I feel comfortable at the moment.
20 I feel chronic fatigue, such as headaches, stiff shoulders, chest tightness, and insomnia.
21 I live a life in which I respect myself.
22 When I want to achieve something, I definitely achieve it.
23 I love myself even when things are not going well for me, e.g., when I am not the person I want to be, such as when I gain a lot of weight, when I do not have money, or when I do not get promoted.
24 There are times when I think that I do not get what I put in in return.
25 I do not get frustrated when results do not turn out the way I want.
26 I have a plan for the future and I work hard every day to make it happen.
27 I am a principal agent who utilizes my time.
28 I sometimes doubt whether my thoughts are right.
29 The process is important even when I do not necessarily achieve my goal.
30 I spend a lot of time working from morning to night.
31 There is someone who unconditionally believes in me and helps me when I have a hard time.
32 There are times when I feel strongly that I have to be nice to others.
33 I know what my own emotions are, so that I control myself in the case where I need to control myself even when I want to enjoy myself, and I also control myself to prepare for the future (the next day's work, etc.).
34 I accept not only good experiences but also painful experiences even when life brings them.
35 I have to become a better person than I am now.
36 I am sacrificial to those close to me.
37 I know why I have to live my life.
38 I do not try excessively hard to maintain good relationships.
39 When a problem arises, I have to overcome it unconditionally.
40 I cannot say what I want to say even when I want to say it.

Among the above items, items 1, 3, 9, 11, 17, 22, 26, 30, 35, and 39 are the items intended to evaluate the degree of “vertical life,” items 4, 8, 12, 15, 19, 21, 25, 29, 34, and 38 are the items intended to evaluate the degree of “horizontal life,” items 5, 10, 13, 16, 20, 24, 28, 32, 36, and 40 are the items intended to evaluate a “gaslighting state,” and items 2, 6, 7, 14, 18, 23, 27, 31, 33, and 37 are the items intended to evaluate an “enlighting state.”

According to one embodiment, when questions for each of the evaluation factors (the vertical life, the horizontal life, the gaslighting state, and the enlighting state) are provided in succession, a user may understand the intention of the questions and enter the values that he or she thinks are ideal regardless of his or her actual thoughts from the user's perspective. Accordingly, as described above, questions related to the individual evaluation factors are presented in a mixed manner. The method of mixing may be a random method, but is not limited thereto. For example, the order of the above 40 questions may change randomly each time they are provided to the user terminal 100.

The question transmission unit 210 may provide an interface that allows the user to enter the degree to which his or her position matches each of the above items, along with the above items. As an example, the question transmission unit 210 may provide an interface that allows the user to enter a value in the range of 1 to 5 for each item.

The response reception unit 220 receives information about the values that are entered for the respective items by the user terminal 100.

The response reception unit 220 receives the values, each in the range of 1 to 5 points, that are entered for the respective items by the user.

The life attitude analysis unit 230 calculates total scores for the respective evaluation factors based on the values for the respective items that are received by the response reception unit 220.

The life attitude analysis unit 230 calculates the total score for the evaluation factor “vertical life” by adding up the values entered for questions 1, 3, 9, 11, 17, 22, 26, 30, 35, and 39 by the user, calculates the total score for the evaluation factor “horizontal life” by adding up the values entered for questions 4, 8, 12, 15, 19, 21, 25, 29, 34, and 38 by the user, calculates the total score for the evaluation factor “gaslighting state” by adding up the values entered for questions 5, 10, 13, 16, 20, 24, 28, 32, 36, and 40 by the user, and calculates the total score for the evaluation factor “enlighting state” by adding up the values entered for questions 2, 6, 7, 14, 18, 23, 27, 31, 33, and 37 by the user.

It may be evaluated that as the total score for the evaluation factor “vertical life” increases, the user has the attitude toward life in which he or she believes that life is valuable only when something is achieved.

It may be evaluated that as the total score for the evaluation factor “horizontal life” increases, the user has the attitude toward life in which he or she accepts and respects himself or herself the way he or she is.

It may be evaluated that as the total score for the evaluation factor “gaslighting state” increases, the user is in the state of mind in which he or she is easily susceptible to gaslighting.

It may be evaluated that as the total score for the evaluation factor “enlighting state” increases, the user is in the state of mind in which he or she can protect himself or herself from gaslighting.

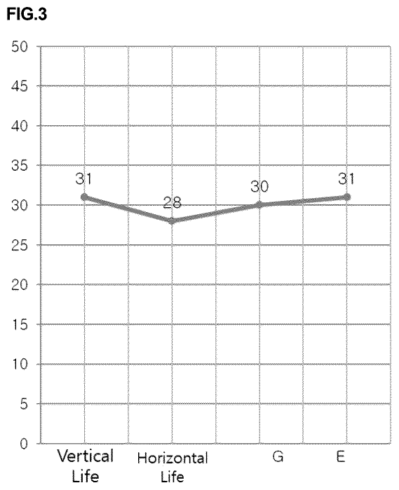

The life attitude analysis unit 230 may provide the user terminal 100 with information about the total scores for the respective evaluation factors in the form of a graph, as shown in FIG. 3.

In FIG. 3, the horizontal axis represents the individual evaluation factors, and G and E represent the gaslighting state and the enlighting state, respectively. Furthermore, the vertical axis represents the total scores for the respective evaluation factors.

Meanwhile, a test result may be one of the following: a first state in which the vertical life has a higher total score than the horizontal life and the gaslighting state has a higher total score than the enlighting state; a second state in which the vertical life has a higher total score than the horizontal life and the gaslighting state has a lower total score than the enlighting state; a third state in which the vertical life has a lower total score than the horizontal life and the gaslighting state has a lower total score than the enlighting state; and a fourth state in which the vertical life has a lower total score than the horizontal life and the gaslighting state has a higher total score than the enlighting state.

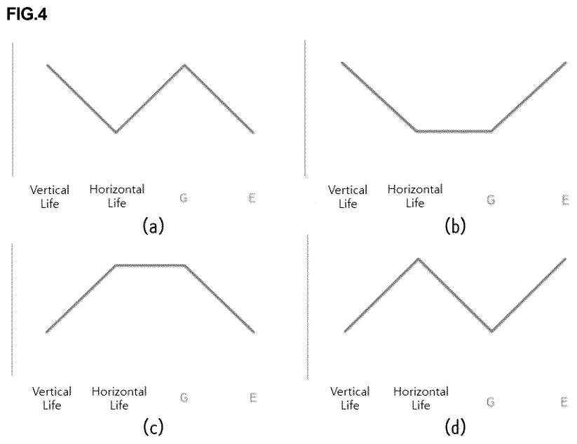

FIG. 4 shows the graph forms of the first to fourth states according to test results according to one embodiment of the present invention.

FIGS. 4(a) to 4(d) respectively represent the graph forms of the first to fourth states.

One of the graphs is provided to the user terminal 100, and a description for the corresponding state is also provided.

In the state of FIG. 4(a), i.e., in the first state, there is provided the description in which this is a state of mind in which a user is easily susceptible to gaslighting, believes that life is valuable (meaningful) only when something is achieved, and has little power to respect or protect oneself.

In the state of FIG. 4(b), i.e., in the second state, there is provided the description in which
a user has the state of mind in which he or she can protect oneself from gaslighting but has the attitude toward life in which he or she believes that life is valuable only when something is achieved.

In the state of FIG. 4(c), i.e., in the third state, there is provided the description in which
a user has the healthiest attitude toward life, has the attitude toward lift in which he or she accepts and respects oneself the way as he or she is, and has the power to protect himself or herself from gaslighting.

In the state of FIG. 4(d), i.e., in the fourth state, there is provided the description in which
a user is in the state of being gaslighted by someone, has no will or desire to escape from gaslighting, and believes that it is okay as it is, so that this is the state in which professional intervention is absolutely required.

In the case of the fourth state, information about a recommended counselor may also be provided to the user terminal 100.

Meanwhile, the life attitude analysis unit 230 transmits a self structure analysis result to the user terminal 100.

First, the total score for the vertical life and the total score for the horizontal life are compared with each other. As a result of the comparison, the name of the evaluation factor having a higher total score (“vertical life” or “horizontal life”) may be transmitted as the self structure analysis result.

Furthermore, the total score for the gaslighting state and the total score for the enlighting state are compared with each other. As a result of the comparison, the name of the evaluation factor having a higher total score (“gaslighting state” or “enlighting state”) may be transmitted as the self structure analysis result.

For example, when the total scores for the respective evaluation factors are 31 points for the vertical life, 28 points for the horizontal life, 30 points for the gaslighting state and 31 points for the enlighting state, “vertical life” and “enlighting state” may be transmitted to the user as the self structure analysis result.

In one embodiment, when the total score for the gaslighting state is high, it may have a negative effect on the achievement of the vertical life. Meanwhile, when the total score for the enlighting state is high, it may have a positive effect on the achievement of the horizontal life.

Accordingly, a first value proportional to the total score G for the gaslighting state is deducted from the total score V for the vertical life, a first value proportional to the total score E for the enlighting state is added to the total score H for the horizontal life, and then the two resulting values are compared to derive a self structure analysis result.

That is, a final score for the vertical life may be (V−K1×G), and a final score for the horizontal life may be (H+K2×E).

Basically, in the case where the vertical life measure and the horizontal life measure are compared with each other, when the total score for the horizontal life measure is higher or the total scores for the two measures are balanced, the user may be interpreted as having a healthy attitude toward life. That is, when the difference between the total score or final score for the vertical life and the total score or final score for the horizontal life is smaller than a preset value, the result of having a healthy attitude toward life may be provided to the user terminal 100.

In addition, the score for the gaslighting state measure and the score for the enlighting state measure are compared with each other, and a higher score for the enlighting state measure may be interpreted as indicating a healthy state of mind in which a user can view and respect himself or herself the way he or she is.

This analysis result may be provided to the user terminal 100.

According to one embodiment, it may be possible to systematically measure the value of a user's life and a self-esteem state in an easy way.

According to one embodiment, it may be possible to evaluate self-protection ability, the degree of satisfaction with life, etc.

The above description of the present invention is intended for illustrative purposes, and those skilled in the art will understand that the present invention may be easily modified into other specific forms without departing from the technical spirit or essential features of the present invention. Therefore, it should be understood that the embodiments described above are exemplary and not limiting in all respects. For example, each component described as a single type may be implemented in a distributed manner, and likewise, components described as distributed may be implemented in a combined form.

The scope of the present invention is defined by the attached claims, and all changed or modified forms derived from the meanings and scope of the claims and their equivalents should be interpreted as being included in the scope of the present invention.

What is claimed is:

1. A life attitude test method, the life attitude test method being performed by a test server, the life attitude test method comprising:
providing a plurality of questions for each of a plurality of evaluation factors, including a vertical life, a horizontal life, a gaslighting state, and an enlighting state, to a user terminal;
receiving user-entered information for each of the plurality of questions for each of the evaluation factors from the user terminal; and
calculating total scores for the respective evaluation factors based on the information received from the user terminal.

2. The life attitude test method of claim 1, wherein providing the plurality of questions for each of the evaluation factors to the user terminal comprises providing the plurality of questions for each of the evaluation factors that are mixed in a random order.

3. The life attitude test method of claim 1, further comprising providing a name of an evaluation factor having a larger one between the total score for the vertical life and the total score for the horizontal life and a name of an evaluation factor having a larger one between the total score for the gaslighting state and the total score for the enlighting state as information about a user's self structure to the user terminal.
